(19) United States
(12) Patent Application Publication
(10) Pub. No.: US 2020/0375788 A1
(43) Pub. Date: Dec. 3, 2020

(54) MENSTRUAL CUP WITH LONGITUDINAL RIBBING

(71) Applicants: Christy J. Zhang, Houston, TX (US); Hanna N. Lauterbach, San Jose, CA (US); Christopher S. Spaulding, Green Bay, WI (US)

(72) Inventors: Christy J. Zhang, Houston, TX (US); Hanna N. Lauterbach, San Jose, CA (US); Christopher S. Spaulding, Green Bay, WI (US)

(21) Appl. No.: 15/931,464

(22) Filed: May 13, 2020

Related U.S. Application Data
(60) Provisional application No. 62/847,187, filed on May 13, 2019.

(51) Int. Cl. A61F 5/455

(52) U.S. Cl. CPC A61F 5/4553

(57) ABSTRACT

A menstrual cup for collecting menstrual fluid. The cup comprises a resilient body further comprising upstanding sidewalls between an open top and a closed bottom and defining an internal cavity for holding the menstrual fluid. The resilient body is biased to an open configuration and has a rim along an edge of the open top. There are spaced-apart longitudinal ribs on one or both of an outer wall surface and/or an inner wall surface of the upstanding sidewalls, and spaced-apart indentations on the inner wall surface. Application of a force at a point of contact on the outer wall surface deforms indentations and the upstanding sidewalls, and thereby releases the menstrual cup from the vaginal canal.

### CROSS-REFERENCE TO RELATED APPLICATIONS

This application claims benefit under 35 U.S.C. § 119(e), of the Provisional Application Assigned Application No. 62/847,187, filed on May 13, 2019, the entire contents of which are hereby incorporated by reference as if fully set forth herein.

### BACKGROUND OF THE INVENTION

Feminine hygiene products, including menstrual cups, are devices worn inside the vagina during menstruation to collect menstrual fluids, which includes menstrual blood, cervical mucus, vaginal secretions, and endometrial tissue. Devices for feminine hygiene and use with menstruation are not new, as described in U.S. Pat. No. 2,534,900 to Chalmers, U.S. Pat. No. 3,845,766 to Zoller, U.S. Pat. No. 3,626,942 to Waldron, and U.S. Pat. No. 5,295,984 to Audrey. Each of these references discloses a cup-shaped menstrual fluid collector made of flexible material inserted into the vaginal canal for the purpose of collecting menstrual blood.

A study shows that insertion and removal of the menstrual cup are significantly harder for new menstrual cup users than new tampon users. Current products on the market, such as the Diva Cup (available from Diva International Inc. of Ontario, Canada), are all functionally similar and all are difficult and messy to remove. Notwithstanding this significant disadvantage for menstrual cups available today, most innovation in this area is currently focused on improving the likelihood of the cup opening after insertion in the vagina, as described in the patents listed above.

Removal of prior art feminine hygiene products is even more difficult and uncomfortable for the user than insertion. The user must reach deep into the vaginal canal with her fingers to push on or near the rim of the menstrual cup to break the seal with the vaginal canal and release the pressure. This release of pressure is also necessary to avoid damaging the internal tissue of the vagina, including the cervix, and without dislodging non biological devices inserted into (and removable from) the vaginal canal, such as an intrauterine device installed by a medically-trained professional.

When using a prior art menstrual cup, displacement of the rim from the vaginal canal may cause a large amount of overflow that travels down the user's fingers and hands, intensifying the discomfort and unpleasantness of this operation.

Korean patent number 1 01 8341 97 B1 discloses an air flow path formed inside the main wall of the menstrual cup to connect the inlet air to the outside air, thereby removing internal negative pressure while breaking the seal to remove the cup.

Published US patent application 2018/0028350 A1 discloses inner ribs in tandem with airholes to release suction from the faceted base.

US issued U.S. Pat. No. 10,357,395 B2 discloses a pull stem extending through the cup to a point on the rim to actuate the side and rim of the cup.

A user's touchpoint for breaking the cup seal, as disclosed by both the Korean patent number 101834197 B1 and the US published patent application 2018/0028350 A1 is proximate to the base of the cup. Additionally, small channels disposed in the cup described in the Korean patent form collection regions for bacteria. Additionally, these regions are difficult to clean. Finally, the cup described in U.S. Pat. No. 10,357,395 requires the user to pull the stem up after removal of the cup to return the cup to an open configuration; this is another difficult and messy process.

For the reasons described, as well as other known problems with prior art menstrual cups, they have not been widely received and adopted by users as an improvement over other feminine hygiene products, such as tampons and pads. The primary problem relates to the difficulties associated with their removal.

Therefore, it is an object of the present invention to provide an advanced menstrual cup that improves wearer comfort and offers a simple and sanitary technique for removal.

### BRIEF DESCRIPTION OF THE DRAWINGS

The present invention is illustrated by way of example, and not by way of limitation, in the figures of the accompanying drawings, in which like reference numerals refer to similar elements and in which:

FIG. 1 illustrates an isometric view of a menstrual cup constructed according to a first embodiment of the present invention.

FIG. 1A illustrates a detailed view of a region of the menstrual cup illustrated in FIG. 1.

FIG. 2 illustrates a top view of a menstrual cup constructed according to another embodiment of the present invention.

FIG. 3 illustrates a side view of a menstrual cup constructed according to another embodiment of the present invention.

FIG. 4 illustrates a cross-sectional side view of a menstrual cup of the present invention in a release conformation.

FIG. 5 illustrates a cross-sectional view of a menstrual cup depicting internal ribs.

FIG. 6 illustrates a side view of a menstrual cup constructed according to yet another embodiment of the present invention.

FIG. 7 illustrates a cross-sectional side view of a menstrual cup according to yet another embodiment of the present invention.

FIG. 8 illustrates a cross-sectional side view of the menstrual cup of FIG. 1.

### DETAILED DESCRIPTION OF THE INVENTION

An apparatus is described for a menstrual cup with longitudinal and/or radial ribbing in a rim of the cup and/or in an inner or outer sidewall of the cup. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5× to 2×, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

The advantages of the present invention will be made more apparent from the following description and drawings. It is understood that changes in the specific structure shown and described may be made within the scope of the claims, without departing from the spirit of the invention.

Advantageously, the menstrual cup of the present invention allows the user to release the seal of the menstrual cup from within the vaginal canal by applying a force at or near a base of the cup.

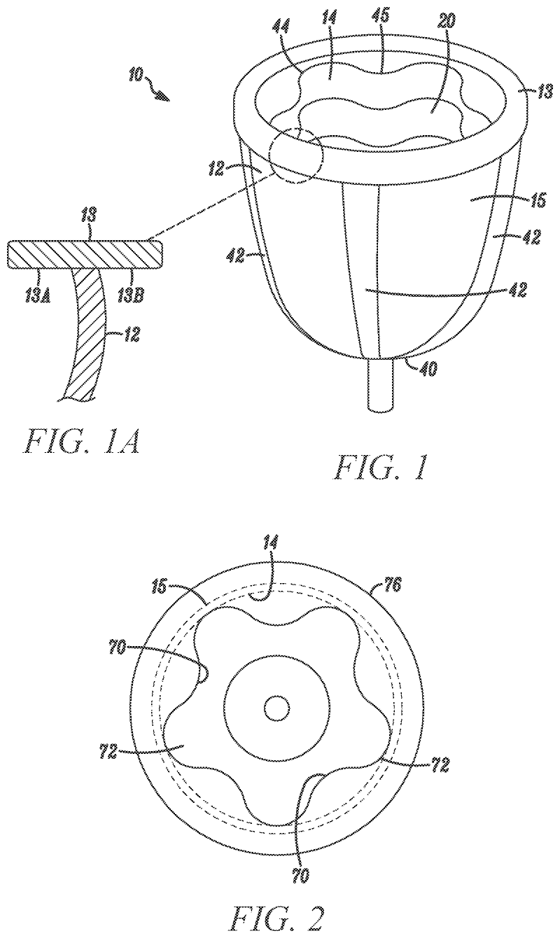

Reference is first made to FIG. 1 to describe a cup 10 in accordance with one embodiment of the invention. The cup 10, indicated in its open conformation in FIG. 1, has a generally inverted conical shape, tapering from a region near a top surface to a curved or rounded bottom surface. In one embodiment, the cup resembles an ice cream cone truncated above the point at the bottom of the cone.

The cup 10 may be regarded as a cylinder tapering toward a lower end and terminating at the lower end with a rounded surface. A tapering sidewall 12 defines an inner wall surface 14 and an outer wall surface 15, thereby forming a cavity 20 for collecting menstrual fluid. The inner and outer wall surfaces 14 and 15 converge to form a closed bottom end 40, which in one embodiment comprises a rounded surface.

As an illustrative non-exclusive example, the internal volume of the cavity 20 may be in a range of 15 to 35 milliliters (mL), but volumes outside of this range are considered within the scope of the present invention.

An annular ring or rim 13 is formed or disposed at an open end of the tapering sidewall 12. According to one embodiment, the rim extends outwardly from the outer wall surface by between about 1 and 2.5 mm and extends inwardly from the inner wall surface by between about 1 and 2.5 mm.

A cross-section detailed illustration FIG. 1A depicts an inner rim segment 13A and an outer rim segment 13B.

A material of the cup comprises a flexible and deformable material, such as medical-grade silicone, for easy insertion and removal. The material is also resilient, permitting the cup to return to its open conformation when placed inside the vagina.

A cup wall thickness is defined as the distance between the inner wall surface 14 and the outer wall surface 15. Generally, the wall thickness near the rim region is greater than the wall thickness at the body of the cup. Preferably, the wall thickness near the rim region is between about 2 millimeters (mm) and 3 mm.

Returning to the embodiment of FIG. 1, the cup 10 includes a plurality of spaced-apart longitudinal external ribs 42 on the outer wall surface 15. The ribs 42 are spaced equidistantly around the outer wall surface 15 when the cup is in the open conformation, extending in a longitudinal direction from a region at or near the rim 13 to a point at or near the closed bottom end 40. The ribs extend from the outer wall surface in a radial direction between about 1 mm and 4 mm.

In an embodiment where the ribs protrude from the inner wall surface (FIG. 5 described below) the ribs also extend radially toward a center of the cup between about 1 mm and 4 mm.

The cumulative length of the ribs (whether disposed on the inner or outer wall surface) is generally at least half a length of the cup extending from the rim 13. However, other embodiments may comprise shorter or longer ribs.

Although various rib configurations are appropriate, FIG. 1 displays one embodiment of the present invention in which the ribs extend along the outer wall surface from the rim 13 to the closed bottom end 40 of the cup 10.

FIG. 1 also depicts indented regions (or indentations) 44 alternating with protruding regions (or protrusions) 45, both formed on the inner wall surface 14. In one embodiment, the protruding regions are about 2 mm to 4 mm in width (i.e., along a circumference of the inner wall) and the indentations are about 4 mm to 10 mm in width (i.e., along a circumference of the inner wall). The protruding regions extend from the inner wall surface between about 2 mm and 5 mm and the indented regions have a depth between about 2 mm and 5 mm.

Generally, an indented region, which is referred to in several of the described embodiments, is defined as a location of a reduced amount of material, i.e., a groove or slot. Similarly, a protruding region is defined as a location with an excess amount of material.

The indented and protruding regions (whether along the inner wall surface, the outer wall surface or the rim) are wavelike in appearance, i.e., a protruding region followed by an indented region followed by a protruding region, etc. Also, the indentations and protrusions do not necessarily extend along the entire vertical length of the cup. In one embodiment the indentations and protrusions extend a distance from the rim and terminate before reaching the closed bottom end 40. In another embodiment the indentations and protrusions extend a distance from the closed bottom surface 40 and terminate before reaching the rim 13.

A cross-sectional view of the cup 10 is depicted in FIG. 8. The indented and protruding regions 44 and 45 extend radially inwardly (an indented region) and outwardly (a protruding region) relative to a vertical centerline of the cup 10 and extend circumferentially around the inner wall surface 14 as shown. The ribs 42 on the outer wall surface 15 are also shown in FIG. 8. The ribs on the outer wall surface can be located opposite an indented region on the inner wall surface or opposite a protruding region on the inner wall surface.

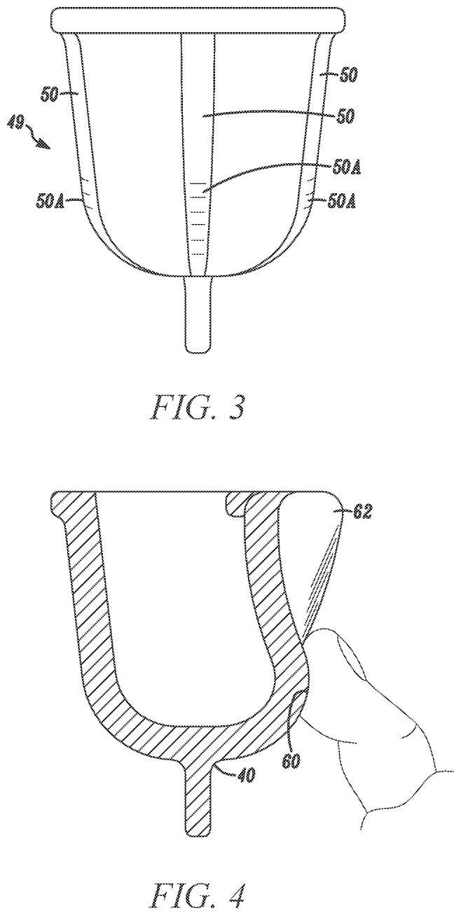

In a FIG. 3 embodiment, a menstrual cup 49 comprises ribs 50 disposed on an outer wall surface 51. The ribs 50 differ from the ribs 42 in the cup 10 of FIG. 1 in that the former comprise textured grip regions 50A.

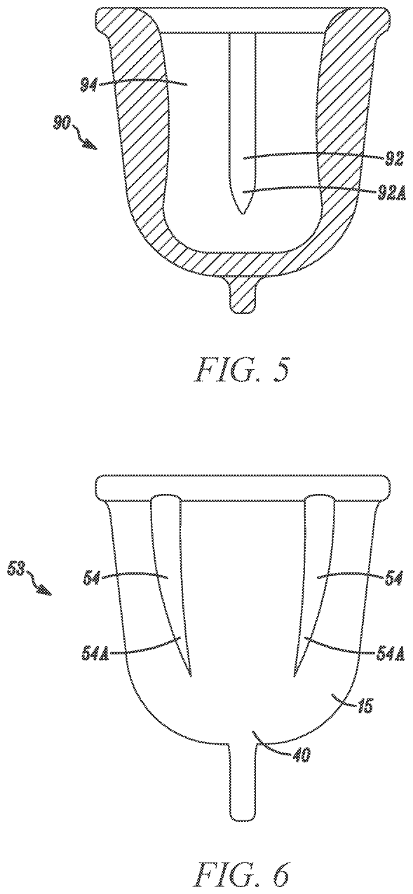

FIG. 6 illustrates a cup 53 with shorter ribs 54 (compared with the ribs 42 of the cup 10 in FIG. 1) on the outer wall surface 15. As shown, the ribs 54 do not extend to the closed bottom end 40 and terminate in a tapered region 54A.

The ribs, indented and protruding regions and the textured grip regions, as illustrated in the various embodiments, aid in removal of the cup 10 from the vagina by transferring forces from a point of contact 60, shown in FIG. 4, to a top region 62 of the cup, thereby deforming the sidewall and the indented regions, so that the seal between the outer wall surface and the vagina is released.

Generally, the point of contact 60 is defined as the lowest point at which the user can apply an inwardly-directed force to the outer wall surface to deform a top region of the cup, thereby breaking the seal with the walls of the vagina. The point of contact 60 is generally within a region extending from about 0.25 inches to about one inch from the closed bottom end 40.

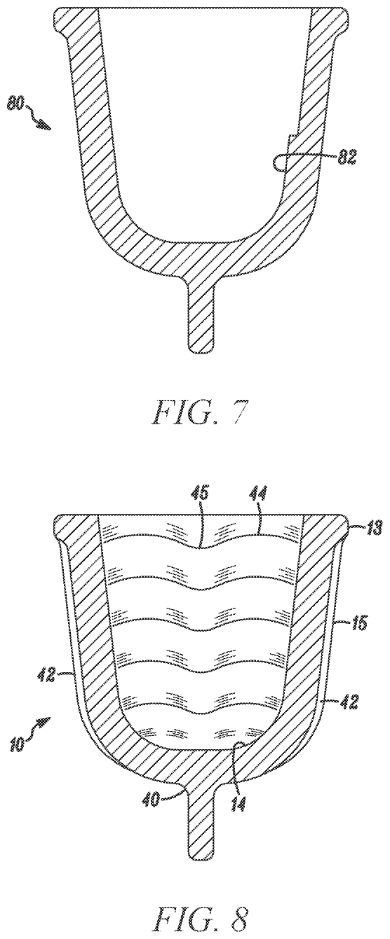

FIG. 7 is a cross-sectional view of another embodiment of a menstrual cup 80 showing internal ribs 82.

A menstrual cup 90 in a cross-section view of FIG. 5, illustrates a rib 92 on an inner surface 94 tapered toward a terminating end 92A. A plurality of ribs 92 extend circumferentially around the inner surface 94.

In yet another embodiment shown in FIG. 2, protruding regions 70 and alternating indented regions 72 are formed only in a rim 76. In this embodiment the rim 76 replaces the rim 13 of FIG. 1. The inner and outer wall surfaces 14 and 15 are shown in phantom in FIG. 2.

Generally, ribs, such as the ribs 42 (FIG. 1), or the ribs 50 (FIG. 3), or the ribs 54 (FIG. 6), can be disposed on the inner wall surface 14 and/or the outer wall surface 15.

Several of the described embodiments have ribs, protrusions and indentations at various locations on either or both of the inner and outer wall surfaces of the cup. Generally, the ribs and protrusions are structural elements that are thicker than the sidewall of the cup. Thus, the rib or protrusion resists bending in response to a force applied to the rib or protrusion. This causes the cup to deform along the indentations or any thinner regions of the sidewall. This action releases the pressure on the vaginal walls at the base of the cup. The user is able to keep her fingers away from the cavity, making the removal process less messy.

While the invention has been described with reference to preferred embodiments, it will be understood by those skilled in the art that various changes may be made and equivalent elements may be substituted for elements thereof without departing from the scope of the present invention. The scope of the present invention further includes any combination of the elements from the various embodiments as set forth herein. In addition, modifications may be made to adapt the teachings of the present invention to a particular application without departing from its essential scope. Therefore, it is intended that the invention not be limited to the particular embodiment disclosed as the best mode contemplated for carrying out this invention nor to the other embodiments described and/or illustrated, but that the invention will include all embodiments falling within the scope of the appended claims.

What is claimed is:

1. A menstrual cup for collecting menstrual fluid in the vaginal canal, comprising:
a resilient body further comprising upstanding and tapering sidewalls between an open top and a closed bottom and defining an internal cavity for holding the menstrual fluid;
the resilient body biased to an open cup-like configuration;
a rim disposed along an edge of the open top;
spaced-apart protrusions disposed on an inner wall surface of the upstanding sidewalls;
spaced-apart indentations formed in the inner wall surface of the upstanding sidewalls, each of the protrusions alternating with an indentation; and
such that application of a force at a point of contact on an outer wall surface deforms the indentations and the upstanding sidewalls for releasing the menstrual cup from within the vaginal canal.

2. The menstrual cup of claim 1, wherein opposing edges defining each one of the indentations are parallel along a first segment of each indentation and converge along a second segment of each indentation.

3. The menstrual cup of claim 2, wherein the second segment is proximate the closed bottom.

4. The menstrual cup of claim 1, wherein each indentation defines an arcuate or a rectangular cross-sectional shape.

5. The menstrual cup of claim 1, wherein the internal cavity defines a volume of between 15 mL and 35 mL.

6. The menstrual cup of claim 1, wherein a thickness of the upstanding sidewalls is between 2 mm and 5 mm

7. The menstrual cup of claim 1, wherein each protrusion is between 2 mm and 4 mm wide and each indentation is between 4 mm and 10 mm wide.

8. The menstrual cup of claim 1, wherein the rim extends radially inwardly toward the internal cavity and radially outwardly beyond the sidewalls.

9. The menstrual cup of claim 1, wherein a contour of the protrusions and indentations is wavelike.

10. The menstrual cup of claim 1, further comprising spaced apart longitudinal ribs on the outer wall surface, wherein the ribs define a semicircular, semi-elliptical, or rectangular cross-sectional shape.

11. The menstrual cup of claim 10, wherein the spaced-apart longitudinal ribs are equally spaced along the outer sidewall and converge at the closed bottom.

12. The menstrual cup of claim 10, wherein each one of the longitudinal ribs is disposed on the outer wall surface and aligned radially outwardly from one of the indentations disposed on the inner wall surface.

13. The menstrual cup of claim 10, wherein opposing edges of each ribs are parallel along an upper segment of each rib and converge along a lower segment of each rib.

14. The menstrual cup of claim 10, wherein a bottom region of each rib defines a textured grip region.

15. The menstrual cup of claim 14, wherein the point of contact is within the textured grip region.

16. The menstrual cup of claim 10, wherein a width of each rib is between 2 mm and 4 mm.

17. The menstrual cup of claim 1, wherein the point of contact is located within a region between about 0.25 inches and one inch from the closed bottom.

18. The menstrual cup of claim 1, wherein the protrusions disposed on the inner wall surface comprise first protrusions, and the indentations formed on the inner wall surface comprise first indentations, wherein second protrusions are disposed on and second indentations are formed in an inwardly facing surface of the rim.

19. A menstrual cup for collecting menstrual fluid, comprising:
a resilient body further comprising upstanding and tapering sidewalls between an open top and a closed bottom and defining an internal cavity for holding the menstrual fluid;
the resilient body biased to an open configuration;
a rim disposed along an edge of the open top;
a plurality of spaced-apart protruding regions disposed on an inner wall surface of the upstanding sidewalls;
a plurality of spaced-apart indented regions on the inner wall surface, each one of the protruding regions alternating with one of the indentations;
spaced-apart longitudinal ribs on an outer wall surface of the upstanding sidewalls, each rib on the outer wall surface radially aligned with an indented region on the inner wall surface; and
such that application of a force at a point of contact on the outer wall surface deforms the indentations and the upstanding sidewalls for releasing the menstrual cup from within the vaginal canal.

20. The menstrual cup of claim 19, wherein a contour of the protruding regions and indented regions is wavelike.
